(19) United States
(12) Patent Application Publication
(10) Pub. No.: US 2009/0013450 A1
(43) Pub. Date: Jan. 15, 2009

(54) Sock

(76) Inventor: Bodo W. Lambertz, Pfaffikon (CH)

Correspondence Address: Olson & Cepuritis, LTD., 20 NORTH WACKER DRIVE, 36TH FLOOR, CHICAGO, IL, 60606, US

(21) Appl. No.: 12/087,397

(22) PCT Filed: Dec. 19, 2006
(86) PCT No.: PCT/DE2006/002269

§ 371 Date: Jul. 3, 2008

(30) Foreign Application Priority Data
Jan. 27, 2006 (DE) 20 2006 001 381.4

(51) Int. Cl. A43B 17/00; A61F 13/00

(52) U.S. Cl. 2/239; 602/65

(57) ABSTRACT

The invention relates to a sock, especially for use in sports activities. Said sock comprises a shaft and a foot part having a toe and a heel area and a contact area between the toe and heel area, and is provided with a bandage. Said bandage (3) is positioned in the area of the ankle joint and consists of at least one web (31) and at least one ring (32).

The invention relates to a sock, in particular for use in athletic activities, with a shaft and a foot part, which has a toe area and a heel area and a tread area between the toe area and the heel area and is provided with a bandage.

In athletic activities in particular, the human feet are usually surrounded by socks. To achieve a good fit of the socks to the foot, it is known that O-ring bandages may be provided on socks or stockings (see U.S. Pat. No. 5,617,745, for example). However, these are designed to run symmetrically and parallel to the central longitudinal line of the sock.

In running and jumping movements, there is increased stress on the foot, in particular in the area of the ankle. The natural function of the foot is to give or roll inward to cushion the impact. This function is called pronation. After standing up on the outside of the sole, the load is shifted slightly toward the inside, so that the longitudinal arch of the foot gives and can thus absorb a portion of the impact. However, the human foot may have different shapes. A distinction is made between a normal foot, talipes caves or hollow foot and pigeon toed as well as the talipes planus or flat foot. A normal foot has a balanced foot curvature. When walking and running, the normal foot comes in contact with the floor only on the outside of the back foot. Then it rolls inward to absorb and dampen the impact of the foot. This is known as natural pronation. Talipes caves or hollow foot and in-toeing or a pigeon toed condition do not primarily pronate inward during the loading phase and leave an impression mainly in the forefoot and the rear foot area. This is known as underpronation and/or supination. The natural impact protection of the foot is greatly reduced in underpronation. A person with flat feet, however, has a very low foot arch and leaves behind a complete foot impression. Flat feet pronate very strongly toward the inside after the ground contact phase. This is known as overpronation. Furthermore, the locomotor system may be stressed in the area of the ankle due to faulty positioning of the legs, usually referred to as bow legs and knock-kneed legs.

Both overpronators and underpronators as well as people with malpositioned legs suffer from the inadequate extent of natural damping. This puts a special load on the foot. To support the tendons and ligaments of the locomotor system around the ankle, it is known that the foot may be bandaged. A bandage is therefore wrapped horizontally around the ankle in the lower area of the shin before putting the sock on the foot.

This wrap offers only an inadequate stabilization and support of the locomotor system around the ankle joint but also does not take into account the specific load forms in overpronation and underpronation. In addition, the bandage under the sock spreads greatly, so that wearing comfort is reduced.

This is where the present invention seeks to remedy the situation. The object of the invention is to create a sock which supports the locomotor system in the area of the ankle and is designed specifically for the special loads in overpronation and underpronation and in malpositioning of the legs. According to the invention, this object is achieved by the fact that the bandage is arranged in the area of the ankle and consists of at least one web and at least one ring.

The present invention creates a sock, in particular for athletic activities, which supports the locomotor system in the area of the ankle and is designed specifically for the special loads that occur in overpronation and underpronation or malpositioning of the legs. For this reason, the bandage runs in the area of the ankle to achieve a supporting function coordinated with the respective load case.

In a further embodiment of the invention, the ring runs asymmetrically on the shaft. This creates support for the ankle in particular and thus an improvement in the supporting effect.

In yet another further embodiment of the invention, the ring runs through the tread area. This creates a fixation effect even beneath the ankle of the foot, which provides additional support for the ankle.

The web advantageously runs over the instep of the foot. Due to this design, there is the possibility of allowing a special fixation in combination with the ring. Here again, good support is achieved in athletic activities.

In addition, the object is achieved by the fact that the bandage consists of two rings which run through the tread area. This creates a sock, in particular for athletic activities, which also supports the locomotor system in the area of the ankle and is designed specifically for the special loads that occur in overpronation and underpronation or malpositioning of the legs. Stronger bandaging is also possible through the use of two combined rings.

The rings in the tread area are preferably placed one above the other. Therefore, double-layer bandaging is possible in the tread area, allowing a further improvement in the supporting function.

Other embodiments and refinements of the invention are characterized in the remaining subclaims. Exemplary embodiments of the invention are depicted in the drawings and are described in detail below, in which:

FIG. 1a) to c) show a sock with a bandage in a view of the outside of the foot as well as a view of a pair of socks from the rear;

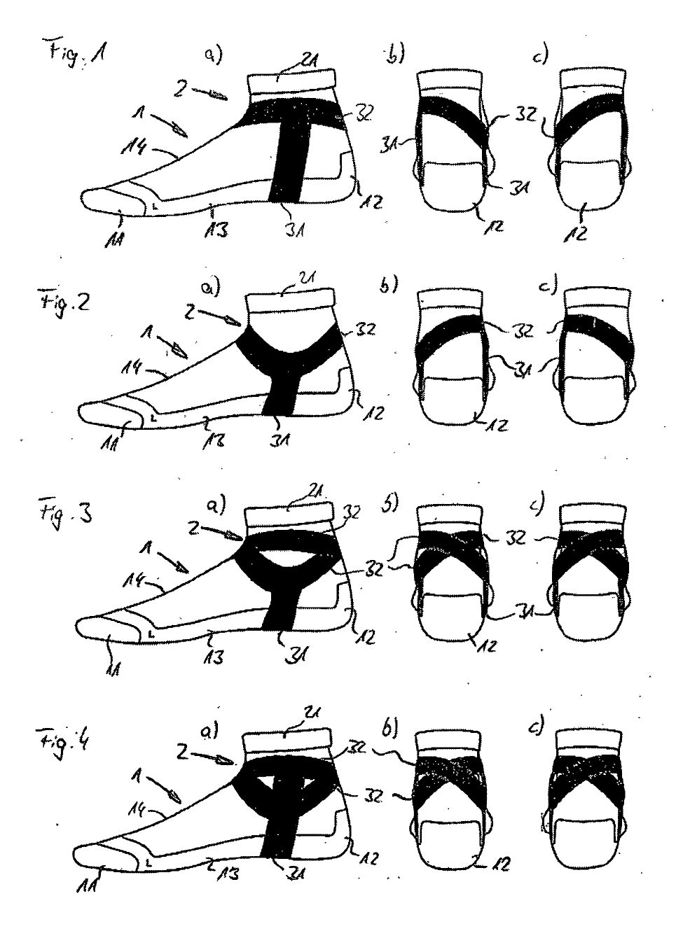

FIG. 2a) to c) show a sock with a bandage in a view of the outside of the foot and a view of a pair of socks from the rear in another embodiment;

FIG. 3a) to c) show a sock with a bandage in the view of the outside of the foot and a view of a pair of socks from the rear in another embodiment;

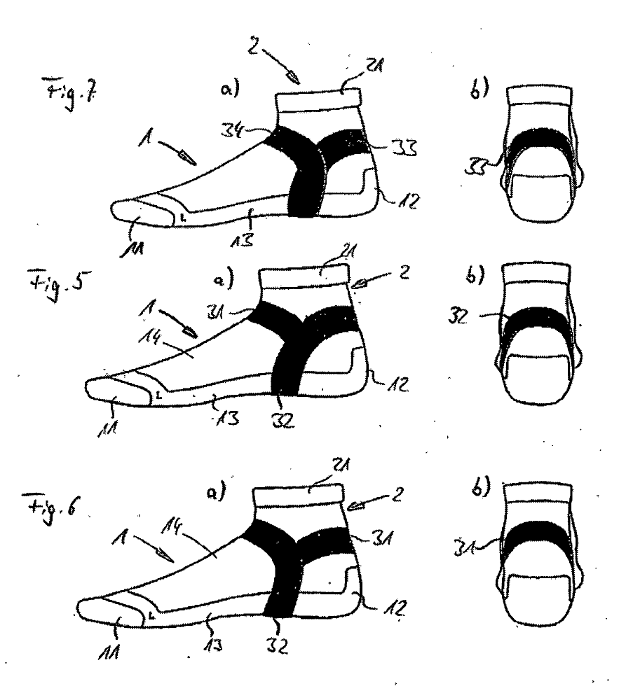

FIG. 4a) to c) show a sock with a bandage in the view of the outside of the foot and a view of a pair of socks from the rear in another embodiment;

FIG. 5a) to b) show a sock with a bandage in the view of the outside of the foot and a view of a pair of socks from the rear in another embodiment;

FIG. 6a) to b) show a sock with a bandage in the view of the outside of the foot and a view of a pair of socks from the rear in another embodiment; and

FIG. 7a) to b) show a sock with a bandage in the view of the outside of the foot and a view of a pair of socks from the rear in another embodiment.

The sock selected as the exemplary embodiment consists of a foot part 1 and a shaft 2. The foot part 1 has a toe area 11, a heel area 12 and a tread area 13 arranged between the toe area and the heel area as well as an instep area 14. The areas 11, 12 and 13 may be made of a reinforced material, as shown in the exemplary embodiment. The use of combinations of materials such as wool with elastomer fiber materials is also possible.

The shaft 2 is provided with a collar 21 on its end facing the foot part 1. In the area of the ankle, the sock is provided with a bandage 3. The bandage 3 is formed from an elastic and climate regulating woven material. Elastan, Lycra or other materials of a different extensibility are preferably used.

The bandage 3 is woven continuously around the sock with the woven fabric that forms the sock. In the exemplary embodiment according to FIGS. 1 through 6, the bandage is formed by a web 31 and a ring 32. The web 31 runs from the ankle “K” to the ankle “K” on the outside by wrapping around the foot.

At the end of the web 31, the ring 32 is attached in the area of the ankle and surrounds the shaft 2 of the sock in the exemplary embodiments according to FIGS. 1 through 6 and essentially runs on its circumference. The web 31 and the ring 32 have the same width and the same material thickness in the exemplary embodiment according to FIGS. 1 through 6.

The ring 32 is arranged asymmetrically. This yields an oblique arrangement of the ring 32 from the upper inside and/or outside in the direction of the lower inside and/or outside in the area of the heel. The ring 32 runs optionally on the outside or inside of the foot above the ankle “K” (FIGS. 1 and 2). In a modification of these exemplary embodiments, it is also possible to protrude [sic; provide] a combination of two rings 32 running asymmetrically (FIGS. 3 and 4). The rings 32 run above and below the ankle “K” on both the outside and inside of the foot. A free space is created between the rings running above and below the ankle, forming a control zone. The ankle is therefore surrounded by the rings, so this further improves the supporting function.

In the exemplary embodiment according to FIGS. 5 and 6, the ring 32 runs through the foot tread area 13. In the exemplary embodiment according to FIG. 5, the ring runs from the tread area 13 over the area of the Achilles tendon. In this way the ring runs around the heel. The web 31 runs in the transition between the instep area 14 and the shaft 2. The free ends of the web 31 are arranged on the ring 32. In the exemplary embodiment according to FIG. 6, a reverse arrangement of the web 31 and ring 32 in comparison with the exemplary embodiment according to FIG. 5 is provided. The web 31 here runs above the heel area 12 in the area of the Achilles tendon; the ring 32 runs around the sock through the line from the tread area to the transition from the instep area 14 and the shaft 2. A further variation in the supporting function is possible through the embodiments according to the exemplary embodiments according to FIGS. 5 and 6.

In the exemplary embodiment according to FIG. 7, the sock also consists of a foot part 1 and a shaft 2. The foot part 1 has a toe area 11, a heel area 12 and a tread area 13 situated between the toe area and the heel area, as well as an instep area 14. The areas 11, 12 and 13 may be made of reinforced material, as shown in the exemplary embodiment. Likewise, the use of combinations of material is also possible. The shaft 2 is provided with a collar 21 on its end facing away from the foot part 1.

In the area of the ankle, the sock is provided with a bandage 3. The bandage 3 consists of two rings 33, 34 which run through the tread area 13. The ring 33 runs from the tread area 13 into the area of the Achilles tendon and thereby surrounds the heel. The ring 34 runs from the tread area 13 to the area of the transition from the heel area 14 and the shaft 2. It thus runs essentially over the forefoot. The rings 33 and 34 are arranged one above the other in the tread area 13. Therefore, this causes either doubling of the material in the area, where one is above the other, or the rings are woven together in the tread area, thus creating a uniform but nevertheless stronger structure. This produces a further increase in the supporting function.

Although the discussion in the description and claims relates to socks, the invention is not limited exclusively to socks but instead this term is also understood to subsume stockings, pantyhose and the like to which the present invention also relates.

1. A sock, in particular for use in athletic activities, having a shaft and a foot part which has a toe area and a heel area and a tread area arranged between the toe area and the heel area and which is provided with a bandage, characterized in that the bandage (3) is arranged in the area of the ankle and comprises at least one web (31) and at least one ring (32), which runs asymmetrically on the circumference of the shaft (2).

2. (canceled)

3. (canceled)

4. A sock according to claim 1, characterized in that the ring (32) runs through the tread area (13).

5. A sock according to claim 1, characterized in that the web (31) runs over the instep area (14).

6. A sock according to claim 1, characterized in that the web (31) runs above the heel area (12).

7. A sock for use in athletic activities, having a shaft and a foot part which has a toe area and a heel area and a tread area arranged between the toe area and the heel area and which is provided with a bandage, characterized in that the bandage (3) consists of two rings (33, 34) which run through the tread area (13) and the rings (33, 34) are arranged one above the other in the tread area (13).

8. A sock according to claim 7, characterized in that the ring (33) surrounds the heel.

9. A sock according to claim 7, characterized in that the rings (34) run over the instep area (14).

10. (canceled)

11. A sock according to claim 2, characterized in that the web (31) runs over the instep area (14).

12. A sock according to claim 4, characterized in that the web (31) runs above the heel area (12).

13. A sock according to claim 5, characterized in that the web (31) runs above the heel area (12).

14. A sock according to claim 8, characterized in that the rings (34) run over the instep area (14).
